(19) United States
(12) Patent Application Publication
(10) Pub. No.: US 2006/0078524 A1
(43) Pub. Date: Apr. 13, 2006

(54) Multi phase personal care composition comprising a conditioning phase and an oil continuous benefit phase

(76) Inventors: Sanjeev Midha, Mason, OH (US); James Merle Heinrich, Fairfield, OH (US); Bryan Gabriel Comstock, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/227,347

(22) Filed: Sep. 15, 2005

Related U.S. Application Data
(60) Provisional application No. 60/617,611, filed on Oct. 8, 2004.

(51) Int. Cl. A61K 8/89; A61K 8/41

(52) U.S. Cl. 424/70.12; 424/70.27; 424/401

(57) ABSTRACT

Multi-phase personal care compositions comprising a fatty compound-cationic surfactant conditioning phase, and at least one benefit phase selected from the group consisting of high internal phase water-in-oil emulsions, hydrophobic gel networks, hydrophobic oil phases, and a silicone or silicone gel. These products are intended to provide a multi-phase personal care composition that is packaged in physical contact while remaining stable and providing improved in-use and after-use hair and skin benefits. The present invention is further directed to a method of using the personal care composition.

### CROSS-REFERENCE TO RELATED APPLICATIONS

This application claims the benefit of U.S. Provisional Application No. 60/617,611 filed on Oct. 8, 2004, the disclosure of which is incorporated herein in its entirety by reference.

### FIELD

The present invention relates to multi-phase personal care compositions suitable for use on mammalian skin and hair. These compositions comprise a conditioning phase and an oil continous benefit phase. These products are intended to provide a multi-phase composition that is packaged in physical contact while remaining stable and providing improved in-use and after-use hair and skin benefits.

### BACKGROUND

A variety of approaches have been used to condition the hair. These range from post-shampooing hair rinses, to leave-on hair conditioners, to inclusion of hair conditioning components in shampoos. Although many consumers prefer the ease and convenience of a shampoo that includes a conditioner, other consumers prefer the more conventional conditioner formulations, which are applied to hair as a separate step from shampooing, usually subsequent to shampooing. These hair conditioners are typically formulated as a single phase, thickened product, such as a gel or cream, for ease of dispensing and application to the hair.

Hair rinse conditioners have conventionally been based on the combination of a cationic surfactant, which is generally a quaternary ammonium compound such as ditallow dimethyl ammonium chloride, and fatty alcohols, such as cetyl and stearyl alcohols. This combination results in a gel-network structure, which provides the composition with a thick, creamy rheology. This thick, creamy rheology is believed to be due a liquid crystalline, lamellar structure formed between cationic surfactants and fatty alcohols. The unique rheology or creaminess of the conditioner cationic surfactant-fatty alcohol gel network is extremely sensitive to the addition of benefit agents. Addition of some benefit agents results in destruction of gel network structure. In addition, deposition of the benefit agents that could be included in the gel matrix may not occur at a desired level required to provide the desired benefits.

It has been long desired to provide consumers multiple benefits from a single product. These benefits include enhanced conditioning, conditioning and styling, hair conditioning and volumizing, conditioning and hair shine enhancement, hair conditioning and coloring, hair conditioning and moisturization, and wet hair conditioning and dry hair conditioning agents.

One attempt at providing multiple benefits from a personal care product has been the dual-chamber packaging. For example, these packages comprise separate compositions and allow for co-dispensing of the two in a single or dual stream. The separate compositions remain physically separate and stable during prolonged storage and just prior to application, then mix during or after dispensing to provide multiple benefits (i.e., conditioning and/or cleansing and/or styling) from a physically stable system. Although such dual-chamber delivery systems provide improved benefits over the use of conventional systems, it is often difficult to achieve consistent and uniform performance because of the uneven dispensing ratio between the two phases from these dual chamber packages. Additionally, these package systems add considerable cost to the finished product.

Accordingly, the need still remains for a personal care composition that provides multiple benefits delivered from one product and remains stable for long periods of time. It is therefore an object of the present invention to provide a multi-phase personal care composition comprising conditioning phases and benefit phases (for example, conditioning, styling, hair shine enhancing, hair coloring, hair moisturizing, hair health enhancing, etc.) that are packaged in physical contact while remaining stable, wherein the compositions provide improved in-use and after-use hair benefits. There also remains a need for delivering weightless conditioning (which does not suppress hair volume and body) from a clear conditioning phase. It remains desirable to provide multiple conditioning benefits from a single product such as delivering weightless conditioning from one phase and more conventional (fatty alcohol/quat gel network with or without silicone) conditioning from an opaque conditioning phase.

### SUMMARY

The present invention is directed to multi-phase personal care compositions comprising a fatty compound-cationic surfactant conditioning phase, and at least one benefit phase selected from the group consisting of high internal phase water-in-oil emulsions, hydrophobic gel networks, hydrophobic oil phases, and a silicone or silicone gel. These products are intended to provide a multi-phase conditioning composition in which two or more phases are packaged in physical contact while remaining stable and providing in-use and after-use hair and skin benefits. The two or more phases in the product are visually distinct and the distinction can be in color or texture or transmittance. The specific patterns can be chosen from a wide variety of patterns, including, but not limited to striping, marbling, geometries, spirals, and mixtures thereof. The composition may optionally comprise additional components providing benefits such as conditioning, styling, coloring, volumizing, shine, health enhancement, and moisturizing. The present invention is further directed to a method of using the personal care composition.

### DETAILED DESCRIPTION

The essential components of the personal care composition are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

By the term “visually distinct,” as used herein, is meant that the regions occupied by each phase can be separately seen by the human eye as distinctly separate regions in contact with one another (i.e. they are not emulsions or dispersions of particles of less than about 100 microns).

By the term “visibly clear” as used herein, is meant that the transmission of the composition is greater than 60%, preferably greater than 80%. The transparency of the composition is measured using Ultra-Violet/Visible (UV/VIS) Spectrophotometry, which determines the absorption or transmission of UV/VIS light by a sample. A light wavelength of 600 nm has been shown to be adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotometer and the percent transmittance is measured by the spectrophotometer at 600 nm.

By the term “multi-phased” or “multi-phase” as used herein, is meant that at least two phases occupy separate and distinct physical spaces inside the package in which they are stored, but are in direct contact with one another (i.e., they are not separated by a barrier and they are not emulsified). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled and combinations thereof. In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The phases may be various different colors, or include particles, glitter or pearlescence.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

The term “water soluble” as used herein, means that the component is soluble in water in the present composition. In general, the component should be soluble at about 25° C. at a concentration of about 0.1% by weight of the water solvent, preferably at about 1%, more preferably at about 5%, even more preferably at about 15%.

The term “anhydrous” as used herein, unless otherwise specified, refers to those compositions or materials containing less than about 10%, more preferably less than about 5%, even more preferably less than about 3%, even more preferably zero percent, by weight of water.

The term “ambient conditions” as used herein, unless otherwise specified, refers to surrounding conditions at one (1) atmosphere of pressure, 50% relative humidity, and 25° C.

The term “stable” as used herein, unless otherwise specified, refers to compositions in which the visible pattern or arrangement of the phases in different locations in the package is not significantly changing overtime when sitting in physical contact at ambient conditions for a period of at least about 180 days. In addition, it is meant that no separation, creaming, or sedimentation occurs. By “separation” is meant that the well-distributed nature of the visually distinct phases is compromised, such that larger regions of at least one phase collect until the balanced dispensed ratio of the two or more compositions relative to each other is compromised.

The term “personal care composition” as used herein, unless otherwise specified, refers to the compositions of the present invention, wherein the compositions are intended to include only those compositions for topical application to the hair or skin, and specifically excludes those compositions that are directed primarily to other applications such as hard surface cleansing, fabric or laundry cleansing, and similar other applications not intended primarily for topical application to the hair or skin.

The present invention relates to multi-phase personal care compositions containing a conditioning phase and a benefit phase suitable for use on mammalian hair or skin. Specifically, the present invention is directed to multi-phase hair or skin conditioning compositions comprising a fatty compound-cationic surfactant conditioning phase, and at least one benefit phase selected from the group consisting of high internal phase water-in-oil emulsions, hydrophobic gel networks, and hydrophobic oil phases that are packaged in physical contact. These compositions can remain stable and can be formulated to provide improved hair benefits during and after application while also providing improved hair conditioning benefits such as softness, smoothness and a clean rinse feel. Further, the compositions of the present invention may deliver weightless conditioning (which does not suppress hair volume and body) from a clear conditioning phase. The present compositions may provide multiple conditioning benefits from a single product such as delivering weightless conditioning from one phase and more conventional (fatty alcohol/quat gel network with or without silicone) conditioning from an opaque conditioning phase. In the present invention, the benefit phase, the conditioning phase, or both the benefit phase and the conditioning phase may be visibly clear.

The compositions of the present invention preferably have a pH of from about 2 to about 8.5, more preferably from about 3 to about 7.5, even preferably from about 3.5 to about 6.5.

The fatty compound-cationic surfactant conditioning phase exhibits a high viscosity, but it is highly shear thinning. The viscosities of the conditioning phase and the benefit phase are in the range of from about 10,000 centipoise to about 200,000,000 centipoise at stress measurements from about 1 to about 20 pascals, more preferably from about 100,000 to about 100,000,000 centipoise at stress measurements from about 1 to about 20 pascals. A Haake RS 150 RheoStress Rheometer may be used to determine the viscosity of the phases. The measurements are made under controlled stress conditions from about 1 pascal to about 500 pascals. A 60 mm parallel plate geometry with a plate gap size of about 0.75 mm is used for measurements. All measurements are taken at about 25° C.

Under appropriate composition, the conditioning phase can form lamellar or vesicle structures. Both lamellar and vesicle structures are considered liquid crystalline and are birefringent. Birefringent materials appear bright between cross-polarizers under an optical microscope.

A. Conditioning Phase

The personal care compositions of the present invention comprise at least one conditioning phase in the form of a fatty compound-cationic surfactant conditioning phase. Preferably, the conditioning phase is present in an amount of from about 1% to about 99%, preferably from about 3% to about 97% by weight of the composition. The conditioning phase includes a cationic surfactant, a fatty compound, and water. The conditioning phase may optionally be in the form of a lamellar, fatty compound-cationic surfactant.

1. Cationic Surfactant

The cationic surfactant is included in the conditioning phase composition at a level by weight of preferably from about 0.1% to about 10%, more preferably from about 1% to about 8%, still more preferably from about 2% to about 5%.

The cationic surfactant, together with below high melting fatty compound, and an aqueous carrier, provides a gel matrix which is suitable for providing various conditioning benefits such as slippery feel on wet hair and softness and moisturized feel on dry hair. In view of providing the above gel matrix, the cationic surfactant and the high melting point fatty compound are contained at a level such that the mole ratio of the cationic surfactant to the high melting point fatty compound is in the range of, preferably from about 1:1 to 1:10, more preferably from about 1:2 to 1:6.

Preferred cationic surfactants are those having a longer alkyl group, i.e., C18-22 alkyl group. Such cationic surfactants include, for example, behenyl trimethyl ammonium chloride and stearyl trimethyl ammonium chloride, and still more preferred is behenyl trimethyl ammonium chloride. It is believed that cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

Among the cationic surfactants useful herein are those corresponding to the general Formula (I): 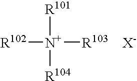
wherein at least one of R¹⁰¹, R¹⁰², R¹⁰³ and R¹⁰⁴ is selected from an aliphatic group of from about 8 to about 30 carbon atoms or an aromatic, alkoxy, polyoxyalkylene, alkylamido, hydroxyalkyl, aryl or alkylaryl group having up to about 22 carbon atoms, the remainder of R¹⁰¹, R¹⁰², R¹⁰³ and R¹⁰⁴ are independently selected from an aliphatic group of from about 1 to about 22 carbon atoms or an aromatic, alkoxy, polyoxyalkylene, alkylamido, hydroxyalkyl, aryl, or alkylaryl group having up to about 22 carbon atoms; X⁻ is a salt-forming anion such as those selected from halogen (e.g., chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages and other groups such as amino groups. The longer chain aliphatic groups, e.g., those of about 12 carbons or higher, can be saturated or unsaturated. Preferred is when R¹⁰¹, R¹⁰², R¹⁰³ and R¹⁰⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general Formula (I), preferred are those containing in the molecule at least one alkyl chain having at least 16 carbons. Nonlimiting examples of such preferred cationic surfactants include: behenyl trimethyl ammonium chloride available with tradename INCROQUAT TMC-80 from Croda and ECONOL TM22 from Sanyo Kasei; cetyl trimethyl ammonium chloride available with tradename CA-2350 from Nikko Chemical, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride.

Also preferred as cationic surfactants are hydrophilically substituted cationic surfactants in which at least one of the substituents contain one or more aromatic, ether, ester, amido, or amino moieties present as substituents or as linkages in the radical chain, wherein at least one of the R¹⁰¹-R¹⁰⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic surfactant contains from about 2 to about 10 nonionic hydrophilic moieties located within the above stated ranges. Preferred hydrophilically substituted cationic surfactants include those of Formulas (II) through (VIII) below:

Formula (II) 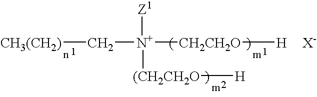
wherein n¹ is from about 8 to about 28, m¹+m² is from about 2 to about 40, Z¹ is a short chain alkyl, preferably a C₁-C₃ alkyl, more preferably methyl, or (CH₂CH₂O)ₘ₃H wherein m¹+m²+m³ is from about 10 to about 60, and X⁻ is a salt-forming anion as defined above;

Formula (III) 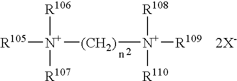
wherein n² is from about 1 to about 5, one or more of R¹⁰⁵, R¹⁰⁶, and R¹⁰⁷ are independently a C₁-C₃₀ alkyl, the remainder are CH₂CH₂OH, one or two of R¹⁰⁸, R¹⁰⁹, and R¹¹⁰ are independently an C₁-C₃₀ alkyl, and the remainder are CH₂CH₂OH, and X⁻ is a salt-forming anion as described above;

Formula (IV)

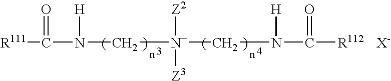

Formula (V) 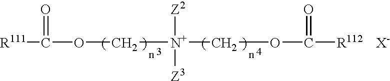
wherein, independently for formulas (IV) and (V), Z² is an alkyl, preferably C₁-C₃ alkyl, more preferably methyl, and Z³ is a short chain hydroxyalkyl (C₁-C₃), preferably hydroxymethyl or hydroxyethyl, n³ and n⁴ independently are integers from about 2 to about 4, inclusive, preferably from about 2 to about 3, inclusive, more preferably 2, R¹¹¹ and R¹¹², independently, are substituted or unsubstituted hydrocarbyls, C₁₂-C₂₀ alkyl or alkenyl, and X⁻ is a salt-forming anion as defined above;

Formula (VI) 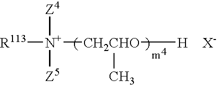
wherein R¹¹³ is a hydrocarbyl, preferably a C₁-C₃ alkyl, more preferably methyl, Z⁴ and Z⁵ are, independently, short chain hydrocarbyls, preferably C₂-C₄ alkyl or alkenyl, more preferably ethyl, m⁴ is from about 2 to about 40, preferably from about 7 to about 30, and X⁻ is a salt-forming anion as defined above;

Formula (VII) 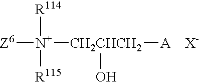
wherein R¹¹⁴ and R¹¹⁵, independently, are C₁-C₃ alkyl, preferably methyl, Z⁶ is a C₁₂-C₂₂ hydrocarbyl, alkyl carboxy or alkylamido, and A is a protein, preferably a collagen, keratin, milk protein, silk, soy protein, wheat protein, or hydrolyzed forms thereof; and X⁻ is a salt-forming anion as defined above;

Formula (VIII) 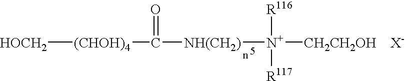
wherein n⁵ is 2 or 3, R¹¹⁶ and R¹¹⁷, independently are C₁-C₃ hydrocarbyls preferably methyl, and X⁻ is a salt-forming anion as defined above. Nonlimiting examples of hydrophilically substituted cationic surfactants useful in the present invention include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemicals, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas.

Salts of primary, secondary, and tertiary fatty amines are also suitable cationic surfactants. The alkyl groups of such amines preferably have from about 12 to about 22 carbon atoms and can be substituted or unsubstituted. Particularly useful are amido substituted tertiary fatty amines. Such amines useful herein include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyl-diethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidylbehenylamine. These amines are typically used in combination with an acid to provide the cationic species. The preferred acid useful herein includes L-glutamic acid, lactic acid, hydrochloric acid, malic acid, succinic acid, acetic acid, fumaric acid, tartaric acid, citric acid, L-glutamic hydrochloride, L-aspartic acid, and mixtures thereof; more preferably L-glutamic acid, lactic acid, and citric acid. Cationic amine surfactants included among those useful in the present invention are disclosed in U.S. Pat. No. 4,275,055.

The molar ratio of protonatable amines to H⁺ from the acid is preferably from about 1:0.3 to 1:1.2, and more preferably from about 1:0.4 to about 1:1.1.

2. Fatty Compound

The conditioning phase composition comprises a fatty compound gel network phase, which consists of from about 0.01% to about 20%, preferably from about 0.1% to about 15%, more preferably from about 0.2% to about 10%, by weight of a fatty compound. A gel matrix may be formed by the fatty compound, and/or the cationic surfactant compound may be first mixed with, suspended in, and/or dissolved in water when forming a gel matrix.

The fatty compound useful herein has a melting point of 25° C. or higher and is selected from the group consisting of fatty alcohols, fatty acids, and mixtures thereof. It is understood that the compounds disclosed in this section of the specification can in some instances fall into more than one classification, e.g., some fatty alcohol derivatives may also be classified as fatty acid derivatives. However, a given classification is not intended to be a limitation on that particular compound, but is done so for the convenience of classification and nomenclature. Further, it is understood that depending on the number and position of double bonds and length and position of branches, certain compounds having certain required carbon atoms may have a melting point of less than 25° C. Such compounds of low melting point are not intended to be included in this section. Nonlimiting examples of high melting compounds are found in International Cosmetic Ingredient Dictionary, Fifth Edition, 1993, and CTFA Cosmetic Ingredient handbook, Second Edition, 1992.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 25 carbon atoms, and more preferably from about 16 to about 22 carbon atoms. These fatty acids are saturated and can be straight or branched chain acids. Also included are diacids, triacids, and other multiple acids that meet the requirements herein. Also included herein are the salts of these fatty acids. Nonlimiting examples of fatty acids include lauric acid, palmitic acid, stearic acid, behenic acid, sebacic acid, and mixtures thereof.

Fatty compounds of a single compound of high purity are preferred. Single compounds of pure fatty alcohols selected from the group of pure cetyl alcohol, stearyl alcohol, and behenyl alcohol are preferred. By “pure” herein, what is meant is that the compound has a purity of at least about 90%, preferably at least about 95%. These single compounds of high purity may provide good rinsability from the hair when the consumer rinses off the composition.

B. Benefit Phase

The personal care compositions of the present invention comprise at least one benefit phase selected from the group consisting of high internal phase water-in-oil emulsions, hydrophobic gel networks, hydrophobic oil phases, and a silicone or silicone gel. Preferably, the benefit phase is present in an amount of from about 1% to about 95%, preferably from about 5% to about 90%, and more preferably from about 10% to about 80% by weight of the composition.

1. High Internal Phase Water-in-Oil Emulsions (HIP)

a. Emulsifier

The water-in-oil emulsions of the present invention may comprise from about 0.01% to about 20% of a low HLB emulsifier, more preferably from about 0.1% to about 10%, still more preferably from about 0.5% to about 9%, of one or more low HLB emulsifier. Preferred low HLB emulsifiers are those having an HLB of from about 1 to about 10, more preferably from 1 to about 8. Suitable low HLB emulsifiers are those selected from saturated C₁₄ to C₃₀ fatty alcohols, saturated C₁₆ to C₃₀ fatty alcohols containing from about 1 to about 5 moles of ethylene oxide, saturated C₁₆ to C₃₀ diols, saturated C₁₆ to C₃₀ monoglycerol ethers, saturated C₁₆ to C₃₀ hydroxy fatty acids, C₁₄ to C₃₀ hydroxylated and nonhydroxylated saturated fatty acids, C₁₄ to C₃₀ saturated ethoxylated fatty acids, amines and alcohols containing from about 1 to about 5 moles of ethylene oxide diols, C₁₄ to C₃₀ saturated glyceryl mono esters with a monoglyceride content of at least about 40%, C₁₄ to C₃₀ saturated polyglycerol esters having from about 1 to about 3 alkyl groups and from about 2 to about 3 saturated glycerol units, C₁₄ to C₃₀ glyceryl mono ethers, C₁₄ to C₃₀ sorbitan mono/diesters, C₁₄ to C₃₀ saturated ethoxylated sorbitan mono/diesters with about 1 to about 5 moles of ethylene oxide, C₁₄ to C₃₀ saturated methyl glucoside esters, C₁₄ to C₃₀ saturated sucrose mono/diesters, C₁₄ to C₃₀ saturated ethoxylated methyl glucoside esters with about 1 to about 5 moles of ethylene oxide, C₁₄ to C₃₀ saturated polyglucosides having an average of between 1 to 2 glucose units and mixtures thereof, having a melting point of at least about 45 degrees C.

The low HLB emulsifiers of the present invention are selected from the group consisting of stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred low HLB emulsifiers of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred low HLB emulsifiers are selected from stearic acid, palmitic acid, stearyl alcohol, cetyl alcohol, behenyl alcohol, steareth-2, and mixtures thereof.

Additional silicone emulsifiers suitable for use herein include the following: Cetyl PEG/PPG-10/1 Dimethicone; Bis-PEG/PPG-14/14 Dimethicone; PEG/PPG-18/18 Dimethicone; PEG-30 Dipolyhydroxystearate; and Laurlyl PEG-9 Polydimethylsiloxyethyl Dimethicone.

Dimethicone copolyol emulsifiers useful herein are described, for example, in U.S. Pat. No. 4,960,764, to Figueroa, Jr. et al., issued Oct. 2, 1990; European Patent No. EP 330,369, to SanoGueira, published Aug. 30, 1989; G. H. Dahms, et al., “New Formulation Possibilities Offered by Silicone Copolyols,” Cosmetics & Toiletries, vol. 110, pp. 91-100, March 1995; M. E. Carlotti et al., “Optimization of W/O-S Emulsions And Study Of The Quantitative Relationships Between Ester Structure And Emulsion Properties,” J. Dispersion Science And Technology, 13(3), 315-336 (1992); P. Hameyer, “Comparative Technological Investigations of Organic and Organosilicone Emulsifiers in Cosmetic Water-in-Oil Emulsion Preparations,” HAPPI 28(4), pp. 88-128 (1991); J. Smid-Korbar et al., “Efficiency and usability of silicone surfactants in emulsions,” Provisional Communication, International Journal of Cosmetic Science, 12, 135-139 (1990); and D. G. Krzysik et al., “A New Silicone Emulsifier For Water-in-Oil Systems,” Drug and Cosmetic Industry, vol. 146(4) pp. 28-81 (April 1990).

Among the non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Other suitable emulsifiers are described, for example, in McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973.

b. Internal Phase Ingredients

The high internal phase water-in-oil emulsions of the present invention may comprise from about 0.1% to about 10% of a thickener, preferably from about 0.25% to about 5%, and more preferably from about 0.5% to about 3%. Preferred thickeners are Pemulen TR-1 (Acrylates/C10-30 Alkyl Acrylate Crosspolymer-Noveon), Pemulen TR-2 (Acrylates/C10-30 Alkyl Acrylate Crosspolymer-Noveon), ETD 2020 (Acrylates/C10-30 Alkyl Acrylate Crosspolymer-Noveon), Carbopol 1382 (Acrylates/C10-30 Alkyl Acrylate Crosspolymer-Noveon), Natrosol CS Plus 330, 430, Polysurf 67 (Cetyl Hydroxyethyl Cellulose-Hercules), Aculyn 22 (Acrylates/Steareth-20 Methacrylate Copolymer-Rohm&Haas) Aculyn 25 (Acrylates/Laureth-25 Methacrylate copolymer-Rohm&Haas), Aculyn 28 (Acrylates/Beheneth-25 Methacrylate copolymer-Rohm&Haas), Aculyn 46 (Peg-150/Stearyl Alcohol/SMDI copolymer-Rohm&Haas) Stabylen 30 (Acrylates/Vinyl Isodecanoate-3V), Structure 2001 (Acrylates/Steareth-20 Itaconate copolymer-National Starch), Structure 3001 (Acrylates/Ceteth-20 Itaconate copolymer-National Starch), Structure Plus (Acrylates/Aminoacrylates/C10-30 Alkyl Peg 20 Itaconate copolymer-National Starch, and Quatrisoft LM-200 (Polyquaternium-24).

The high internal phase water-in-oil emulsions of the present invention may comprise natural and synthetic clays such as Laponite XLS (Southern Clay Products). In addition, the high internal phase water-in-oil emulsions may also comprise various salts, glycerin and its derivatives, and a variety of benefit agents described under the optional ingredient section.

c. Hydrophobic Oils

The hydrophobic oil or blend of oils is present in both the water-in-oil emulsions and the hydrophobic gel described below. The benefit phase comprising water-in-oil emulsions of the present composition may comprise from about 1% to about 99% of oil, more preferably from about 3% to about 60% of oil, even more preferably from about 5% to about 30% of oil, and still more preferably from about 10% to about 20% of oil. When the amount of oil present is less than 25%, the emulsions are characterized as high internal phase emulsions. The oils suitable for use in water-in-oil emulsions are liquid at ambient conditions.

Oils suitable for use herein include any natural and synthetic materials with an overall solubility parameter less than about 12.5 (cal/cm³)^{0.5}, preferably less than about 11.5 (cal/cm³)^{0.5}. Solubility parameters for the oils described herein are determined by methods well known in the chemical arts for establishing the relative polar character of a material. A description of solubility parameters and means for determining them are described by C. D. Vaughn, “Solubility Effects in Product, Package, Penetration and Preservation” 103 Cosmetics and Toiletries 47-69, October 1988; and C. D. Vaughn, “Using Solubility Parameters in Cosmetics Formulation”, 36 J. Soc. Cosmetic Chemists 319-333, September/October, 1988.

The oil present in the composition has a Vaughan Solubility Parameter (VSP) of from about 5 to about 10, preferably from about 6 to less than 10, more preferably from about 6 to about 9. Non-limiting examples of benefit agents having VSP values ranging from about 5 to about 10 include the following:

Vaughan Solubility Parameter*

| | |
|---|---|
| Cyclomethicone D5 | 5.77 |
| Dimethicone | 5.92 |
| Squalane | 6.03 |
| Petrolatum | 7.33 |
| Isopropyl Palmitate | 7.78 |
| Isopropyl Myristate | 8.02 |
| Castor Oil | 8.90 |
| Cholesterol | 9.55 |

* As reported in Solubility, Effects in Product, Package, Penetration and Preservation, C. D. Vaughan, Cosmetics and Toiletries, Vol. 103, October 1988.

As used herein, “overall solubility parameter” means that it is possible to use oils with higher solubility parameters than 12.5 (cal/cm³)^{0.5} if they are blended with other oils to reduce the overall solubility parameter of the oil mixture to less than about 12.5 (cal/cm³)^{0.5}. For example, a small portion of diethylene glycol (sol par=13.61) could be blended with lanolin oil (sol par=7.3) and a cosolublizing agent to create a mixture that has a solubility parameter of less than about 12.5(cal/cm³)^{0.5}.

Oils suitable for use herein include, but are not limited to, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin and its derivatives, wax esters, beeswax derivatives, sterols and phospholipids, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Non-limiting examples of silicone oils suitable for use herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof. In addition, any of the above oils that have been partially or fully hydrogenated are also suitable.

Non-limiting examples of acetoglyceride esters suitable for use herein include acetylated monoglycerides.

Non-limiting examples of alkyl esters suitable for use herein include isopropyl esters of fatty acids and long chain esters of long chain fatty acids, e.g. SEFA (sucrose esters of fatty acids). Lauryl pyrolidone carboxylic acid, pentaerthritol esters, aromatic mono, di or triesters, cetyl ricinoleate, non-limiting examples of which include isopropyl palmitate, isopropyl myristate, cetyl riconoleate and stearyl riconoleate. Other examples include, but are not limited to, hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use herein include oleyl myristate, oleyl stearate, oleyl oleate, and combinations thereof.

Non-limiting examples of lanolin and lanolin derivatives suitable for use herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, hydroxylated lanolin, hydrogenated lanolin and combinations thereof.

Still other suitable oils include milk triglycerides (e.g., hydroxylated milk glyceride) and polyol fatty acid polyesters.

Still other suitable oils include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

2. Hydrophobic Gel

Another embodiment of the present invention may comprise a hydrophobic gel. The oils described for use in water-in-oil emulsions are suitable for use as the benefit phase as long as they meet the viscosity requirements.

Many hydrophobic gels are based on a variety of hydrocarbons and esters. The gellants are combinations of an ethylene/propylene/styrene copolymer and a butylene/ethylene/styrene copolymer. Various gelled hydrocarbon solvents can be used to deliver conditioning ingredients onto hair surface. Hydrocarbon solvents can be volatile or non-volatile. The hydrophobic gel network may comprise hydrophobic solvents thickened with polymeric gelling agents. Suitable hydrocarbon gels are available under the trade name Versagel by the Penereco Corporation.

Examples of non-volatile solvent based gels are Versagel materials including Versagel M (mineral oil based), Versagel ME (hydrogenated polyisobutene based), Versagel MP (isopropyl palmitate based), Versagel MC (isohexadecane based). An example of a volatile hydrocarbon gel is Versagel MD (isododecane based).

Other examples of hydrophobic gels are high molecular weight silicone gums, elastomers, and their blends. Examples are Dow Corning 200 fluids (60000, 300000, and 600000cs), Dow Corning 9040 Silicone Elastomers.

3. Silicone or Silicone Gel

Another embodiment of the present invention may comprise a silicone or silicone gel. The silicones described for use in water-in-oil emulsions are suitable for use in the benefit phase as long as they meet the viscosity requirements. High molecular weight silicones and silicone gums can be used as they have inherent conditioning on hair. Examples of high molecular weight dimethicone are Dow Corning 200 fluids (60000, 300000, and 600000 cst). Low molecular weight silicones can be gelled, added to high molecular weight silicones, or a combination of both. Examples of suitable silicone gellants are silicone elastomers such as Dow Corning 9040. The silicones can be volatilve or non-volatile, with the preferred silicone dependent on the desired benefit.

C. Aqueous Carrier

The compositions of the present invention may comprise an aqueous carrier. The aqueous carrier may be found in the conditioning phase, the benefit phase, or both the conditioning phase and the benefit phase. Preferably, they comprise from about 50% to about 99.8%, by weight of water. The aqueous carrier can optionally include other liquid, water-miscible or water-soluble solvents such as lower alkyl alcohols, e.g. C₁-C₅ alkyl monohydric alcohols, preferably C₂-C₃ alkyl alcohols. However, the fatty compound must be miscible in the aqueous carrier of the composition. The fatty compound can be naturally miscible in the aqueous carrier or can be made miscible through the use of cosolvents or surfactants.

D. Additional Components

The compositions of the present invention may comprise additional components. The additional components may be found in the conditioning phase, the benefit phase, or both the conditioning phase and the benefit phase.

1. Humectants and Solutes

A suitable benefit agent is one or more humectants and solutes. A variety of humectants and solutes can be employed and can be present at a level of from about 0.1% to about 50%, preferably from about 0.5% to about 35%, and more preferably from about 2% to about 20% by weight of a non-volatile, organic material having a solubility of at least 5 parts in 10 parts water. A preferred water soluble, organic material is selected from the group consisting of a polyol of the structure:
R1-O(CH₂—CR2O)ₙH
where R1=H, C1-C4 alkyl; R2=H, CH₃ and n=1-200; C₂-C₁₀ alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)ₓNH_{y} where x=1-3; y=0-2, and x+y=3, and mixtures thereof. Preferred polyols are selected from the group consisting of glycerine, polyoxypropylene(1) glycerol and polyoxypropylene(3) glycerol, sorbitol, butylene glycol, propylene glycol, sucrose, urea and triethanol amine.

2. Water Soluble Nonionic Polymers

The compositions of the present invention may comprise from about 0.1% to about 10%, more preferably from about 0.2% to about 5%, and even more preferably from about 0.5% to about 3% by weight of a water soluble nonionic polymer.

The polymers of the present invention are characterized by the general formula: 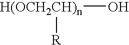
wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, n has an average value of from about 2,000 to about 14,000, preferably from about 5,000 to about 9,000, more preferably from about 6,000 to about 8,000.

Polyethylene glycol polymers useful herein that are especially preferred are PEG-2M wherein R equals H and n has an average value of about 2,000 (PEG 2-M is also known as Polyox WSR® N-10 from Union Carbide and as PEG-2,000); PEG-SM wherein R equals H and n has an average value of about 5,000 (PEG 5-M is also known as Polyox WSR® N-35 and Polyox WSR® N-80, both from Union Carbide and as PEG-5,000 and Polyethylene Glycol 300,000); PEG-7M wherein R equals H and n has an average value of about 7,000 (PEG 7-M is also known as Polyox WSR® N-750 from Union Carbide); PEG-9M wherein R equals H and n has an average value of about 9,000 (PEG 9-M is also known as Polyox WSR® N-3333 from Union Carbide); and PEG-14 M wherein R equals H and n has an average value of about 14,000 (PEG 14-M is also known as Polyox WSR® N-3000 from Union Carbide.) Other useful polymers include the polypropylene glycols and mixed polyethylene/polypropylene glycols.

3. Hair Coloring Agents/Dyes

The compositions of the present invention may also include hair coloring agents/dyes. Hair coloring agents/dyes useful herein include anthroquinone, azo, nitro, basic, triarylmethane, or disperse dyes, or any combinations thereof. A range of direct dyes, including basic dyes and neutral dyes are useful herein. Dyes suitable for use are described in U.S. Pat. No. 5,281,240 and U.S. Pat. No. 4,964,874.

4. Cationic Polymer Conditioning Agent

The compositions of the present invention can also comprise one or more cationic polymer conditioning agents. The cationic polymer conditioning agents will preferably be water soluble. Cationic polymers are typically used in the same ranges as disclosed above for cationic surfactants. The cationic polymer conditioning agents may also be useful as thickeners as described herein.

By “water soluble” cationic polymer, what is meant is a polymer which is sufficiently soluble in water to form a substantially clear solution to the naked eye at a concentration of 0.1% in water (distilled or equivalent) at 25° C. Preferably, the polymer will be sufficiently soluble to form a substantially clear solution at 0.5% concentration, more preferably at 1.0% concentration.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/gram, more preferably at least about 0.5 meq/gram, even more preferably at least abut 1.1 meq/gram, even more preferably at least about 1.2 meq/gram. The average molecular weight of such suitable cationic polymers will generally be between about 10,000 and about 10 million, preferably between about 50,000 and about 5 million, more preferably between about 100,000 and about 3 million. Those skilled in the art will recognize that the charge density of amino-containing polymers may vary depending upon pH and the isoelectric point of the amino groups. The charge density should be within the above limits at the pH of intended use.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from about 3 to about 5 carbon atoms, as described in U.S. Pat. No. 4,009,256.

Other cationic polymers that can be used include polysaccharide polymers, such as cationic cellulose derivatives and cationic starch derivatives.

Cationic polysaccharide polymer materials suitable for use herein include those of the formula: 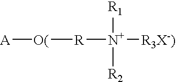
wherein: A is an anhydroglucose residual group, such as a starch or cellulose anhydroglucose residual, R is an alkylene oxyalkylene, polyoxyalkylene, or hydroxyalkylene group, or combination thereof, R₁, R₂, and R₃ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R₁, R₂ and R₃) preferably being about 20 or less, and X is an anionic counterion. Suitable counterions include halides (e.g., Cl, Br, I, or F, preferably Cl, Br, or I), sulfate, and methylsulfate. Others can also be used, as this list is not exclusive.

Cationic cellulose is available from Amerchol Corp. in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted opoxide, referred to in the industry (CTFA) as Polyquaternium 24. These materials are available from Amerchol Corp. under the tradename Polymer LM-200®.

Other cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418), and copolymers of etherified cellulose and starch (e.g., as described in U.S. Pat. No. 3,958,581).

As discussed above, the cationic polymer hereof is water soluble. This does not mean, however, that it must be soluble in the composition. Preferably however, the cationic polymer is either soluble in the composition or in a complex coacervate phase in the composition formed by the cationic polymer and anionic material. Complex coacervates of the cationic polymer can be formed with anionic surfactants or with anionic polymers that can optionally be added to the compositions hereof (e.g., sodium polystyrene sulfonate).

5. Silicone Conditioning Agents

The compositions hereof can also include nonvolatile soluble or insoluble silicone conditioning agents. By soluble what is meant is that the silicone conditioning agent is miscible with the aqueous carrier of the composition so as to form part of the same phase. By insoluble what is meant is that the silicone forms a separate, discontinuous phase from the aqueous carrier, such as in the form of an emulsion or a suspension of droplets of the silicone.

The silicone hair conditioning agent will be used in the compositions hereof at levels of from about 0.05% to about 10% by weight of the composition, preferably from about 0.1% to about 6%, more preferably from about 0.3% to about 5%, even more preferably from about 0.5% to about 3%.

Soluble silicones include silicone copolyols, such as dimethicone copolyols, e.g. polyether siloxane-modified polymers, such as polypropylene oxide, polyethylene oxide modified polydimethylsiloxane, wherein the level of ethylene and/or propylene oxide is sufficient to allow solubility in the composition.

Preferred, however, are insoluble silicones. The insoluble silicone hair conditioning agent for use herein will preferably have viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Corning Corporate Test Method CTM0004, Jul. 20, 1970.

Suitable insoluble, nonvolatile silicone fluids include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. The term “nonvolatile” as used herein shall mean that the silicone has a boiling point of at least about 260° C., preferably at least about 275° C., more preferably at least about 300° C. Such materials exhibit very low or no significant vapor pressure at ambient conditions. The term “silicone fluid” shall mean flowable silicone materials having a viscosity of less than about 1,000,000 centistokes at 25° C. Generally, the viscosity of the fluid will be between about 5 and about 1,000,000 centistokes at 25° C., preferably between about 10 and about 300,000 centistokes.

Silicone fluids hereof also include polyalkyl or polyaryl siloxanes with the following structure: 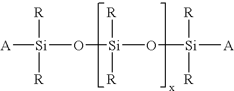
wherein R is alkyl or aryl, and x is an integer from about 7 to about 8,000. “A” represents groups which block the ends of the silicone chains.

The alkyl or aryl groups substituted on the siloxane chain (R) or at the ends of the siloxane chains (A) may have any structure as long as the resulting silicones remain fluid at room temperature, are hydrophobic, are neither irritating, toxic nor otherwise harmful when applied to the hair, are compatible with the other components of the composition, are chemically stable under normal use and storage conditions, and are capable of being deposited on and conditioning hair.

Suitable A groups include methyl, methoxy, ethoxy, propoxy, and aryloxy. The two R groups on the silicone atom may represent the same group or different groups. Preferably, the two R groups represent the same group. Suitable R groups include methyl, ethyl, propyl, phenyl, methylphenyl, and phenylmethyl. The preferred silicones are polydimethyl siloxane, polydiethylsiloxane, and polymethylphenylsiloxane. Polydimethylsiloxane is especially preferred.

The nonvolatile polyalkylsiloxane fluids that may be used include, for example, polydimethylsiloxanes. These siloxanes are available, for example, from the General Electric Company in their ViscasilR and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

Especially preferred for enhancing the shine characteristics of hair are highly arylated silicones, such as highly phenylated polyethyl silicone having refractive indices of about 1.46 or higher, especially about 1.52 or higher. When these high refractive index silicones are used, they should be mixed with a spreading agent such as a surfactant or a silicone resin, as described below, to decrease the surface tension and enhance the film forming ability of the material.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level should be sufficiently low to prevent solubility in the composition hereof.

References disclosing suitable silicone fluids include U.S. Pat. No. 2,826,551; U.S. Pat. No. 3,964,500; U.S. Pat. No. 4,364,837; and British Patent 849,433. Silicon Compounds distributed by Petrarch Systems, Inc., 1984, provides an extensive (though not exclusive) listing of suitable silicone fluids.

Another silicone hair conditioning material that can be especially useful in the silicone conditioning agents is insoluble silicone gum. The term “silicone gum”, as used herein, means polyorganosiloxane materials having a viscosity at 25° C. of greater than or equal to 1,000,000 centistokes. Silicone gums are described by Petrarch and others including U.S. Pat. No. 4,152,416 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a mass molecular weight in excess of about 200,000, generally between about 200,000 and about 1,000,000. Specific examples include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

Preferably the silicone hair conditioning agent comprises a mixture of a polydimethylsiloxane gum, having a viscosity greater than about 1,000,000 centistokes and polydimethylsiloxane fluid having a viscosity of from about 10 centistokes to about 100,000 centistokes, wherein the ratio of gum to fluid is from about 30:70 to about 70:30, preferably from about 40:60 to about 60:40.

An optional ingredient that can be included in the silicone conditioning agent is silicone resin. Silicone resins are highly crosslinked polymeric siloxane systems. The crosslinking is introduced through the incorporation of trifunctional and tetrafunctional silanes with monofunctional or difunctional, or both, silanes during manufacture of the silicone resin. As is understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Silicone resins can enhance deposition of silicone on the hair and can enhance the glossiness of hair with high refractive index volumes.

Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight, complete the description of the silicone material under the MDTQ system. Higher relative molar amounts of T, Q, T′ and/or Q′ to D, D′, M and/or M′ in a silicone resin is indicative of higher levels of crosslinking. However, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

6. Anti-Dandruff Agents

The compositions of the present invention may also contain an anti-dandruff agent. Suitable, non-limiting examples of anti-dandruff particulates include: pyridinethione salts, azoles, selenium sulfide, climbazole, particulate sulfur, and mixtures thereof. Preferred are pyridinethione salts. Such anti-dandruff particulate should be physically and chemically compatible with the essential components of the composition, and should not otherwise unduly impair product stability, aesthetics or performance.

Pyridinethione anti-dandruff particulates, especially 1-hydroxy-2-pyridinethione salts, are highly preferred particulate anti-dandruff agents for use in compositions of the present invention. The concentration of pyridinethione anti-dandruff particulate typically ranges from about 0.1% to about 4%, by weight of the composition, preferably from about 0.1% to about 3%, more preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form, wherein the particles have an average size of up to about 20μ, preferably up to about 5μ, more preferably up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller.

In addition to the anti-dandruff active selected from polyvalent metal salts of pyrithione, the present invention may further comprise one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulphide and coal tar.

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

Selenium sulfide is a particulate anti-dandruff agent suitable for use in the anti-microbial compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof sulfur are typically from about 1% to about 4%, preferably from about 2% to about 4%.

7. Particles

The personal care composition of the present invention may comprise particles. Water insoluble solid particle of various shapes and densities is useful. The particle of the present invention has a particle size (volume average based on the particle size measurement described hereafter) of less than about 100 μm, preferably less than about 60 μm, and more preferably the particle size of less than about 30 μm.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

The interference pigments of the present invention are platelet particulates. The platelet particulates of the multi-phased personal care compositions preferably have a thickness of no more than about 5 μm, more preferably no more than about 2 μm, still more preferably no more than about 1 μm. The platelet particulates of the multi-phased personal care composition preferably have a thickness of at least about 0.02 μm, more preferably at least about 0.05 μm, even more preferably at least about 0.1 μm, and still more preferably at least about 0.2 μm.

The interference pigment of the multi-phased personal care compositions comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃SnO₂. For the multiple layer structures, the thin films can be consisted of all high refractive index materials or alternation of thin films with high and low RI materials with the high RI film as the top layer.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The Particle Contact Angle Test as described in copending application Ser. No. 60/469,075 filed on May 8, 2003 is used to determine contact angle of interference pigments. The greater the contact angle, the greater the hydrophobicity of the interference pigment. The interference pigment of the present invention possess a contact angle of at least 60 degrees, more preferably greater than 80 degrees, even more preferably greater than 100 degrees, still more preferably greater than 100 degrees.

Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

8. Styling Polymers

The compositions of the present invention may comprise styling polymers. The compositions hereof will generally comprise from about 0.1% to about 15%, preferably from 0.5% to about 8%, more preferably from about 1% to about 8%, by weight of the composition, of the styling polymer. It is not intended to exclude the use of higher or lower levels of the polymers, as long as an effective amount is used to provide adhesive or film-forming properties to the composition and the composition can be formulated and effectively applied for its intended purpose.

These styling polymers provide the composition of the present invention with hair styling performance by providing polymeric deposits on the hair after application. The polymer deposited on the hair has adhesive and cohesive strength and delivers styling primarily by forming welds between hair fibers upon drying, as is understood by those skilled in the art.

Many such polymers are known in the art, including water-soluble and water-insoluble organic polymers and water-insoluble silicone-grafted polymers, all of which are suitable for use in the composition herein, provided that they also have the requisite features or characteristics described hereinafter. Such polymers can be made by conventional or otherwise known polymerization techniques well known in the art, an example of which includes free radical polymerization.

The styling polymer should have a weight average molecular weight of at least about 20,000, preferably greater than about 25,000, more preferably greater than about 30,000, most preferably greater than about 35,000. There is no upper limit for molecular weight except that which limits applicability of the invention for practical reasons, such as processing, aesthetic characteristics, ability to formulate, etc. In general, the weight average molecular weight will be less than about 10,000,000, more generally less than about 5,000,000, and typically less than about 2,000,000. Preferably, the weight average molecular weight will be between about 20,000 and about 2,000,000, more preferably between about 30,000 and about 1,000,000, and most preferably between about 40,000 and about 500,000.

Suitable silicone grafted polymers are also disclosed in EPO Application 90307528.1, published as EPO Application 0 408 311 A2 on Jan. 11, 1991, Hayama, et al., U.S. Pat. No. 5,061,481, issued Oct. 29, 1991, Suzuki et al., U.S. Pat. No. 5,106,609, Bolich et al., issued Apr. 21, 1992, U.S. Pat. No. 5,100,658, Bolich et al., issued Mar. 31, 1992, U.S. Pat. No. 5,100,657, Ansher-Jackson, et al., issued Mar. 31, 1992, U.S. Pat. No. 5,104,646, Bolich et al., issued Apr. 14, 1992, U.S. Ser. No. 07/758,319, Bolich et al, filed Aug. 27, 1991, and U.S. Ser. No. 07/758,320, Torgerson et al., filed Aug. 27, 1991.

Suitable cationic polymers include Polyquaternium-4 (Celquat H-100; L200—supplier National Starch); Polyquaternium-10 (Celquat SC-240C; SC-230 M—supplier National Starch); (UCARE polymer series—JR-125, JR-400, LR-400, LR-30M, LK, supplier Amerchol); Polyquaternium-11 (Gafquat 734; 755N—supplier ISP); Polyquaternium-16 (Luviquat FC 370; FC550; FC905; HM-552 supplier by BASF); PVP/Dimethylaminoethylmethacrylate (Copolymer 845; 937; 958-ISP supplier); Vinyl Caprolactam/PVP/Dimethylaminoethyl Methacrylate copolymer (Gaffix VC-713; H2 OLD EP-1—supplier ISP); Chitosan (Kytamer L; Kytamer PC—supplier Amerchol); Polyquaternium-7 (Merquat 550—supplier Calgon); Polyquaternium-18 (Mirapol AZ-1 supplied by Rhone-Poulenc); Polyquaternium-24 (Quatrisoft Polymer LM-200—supplier Amerchol); Polyquaternium-28 (Gafquat HS-100—supplier ISP); Polyquaternium-46 (Luviquat Hold—supplier BASF); and Chitosan Glycolate (Hydagen CMF; CMFP—supplier Henkel); Hydroxyethyl Cetyldimonium Phosphate (Luviquat Mono CP—supplier BASF); and Guar Hydroxylpropyl Trimonium Chloride (Jaguar C series -13S, -14S, -17, 162,-2000, Hi-CARE 1000—supplier Rhône-Poulenc).

Suitable amphoteric polymers include Octylacrylmide/Acrylates/Butylaminoethyl Methacrylate Copolymer (Amphomer 28-4910, Amphomer LV-71 28-4971, Lovocryl-47 28-4947—National Starch supplier), and Methacryloyl ethyl betaine/methacrylates copolymer (Diaformer series supplier Mitsubishi).

Polymers which are partially zwitterionic are also useful. They possess a positive charge over a broad range of pH but contain acidic groups which are only negatively charged at basic pH. The polymer is positively charged at lower pH and neutral (have both negative and positive charge) at higher pHs. The zwitterionic polymer may be selected from cellulose derivatives, wheat derivatives and chitin derivatives such as are known in the art. Nonlimiting examples of zwitterionic polymers useful herein include Polyquaternium-47 (Merquat 2001—supplier Calgon (a zwitterionic copolymer of acrylic acid, methacryl amido propyl trimethyl ammonium chloride, and methyl acrylate)); Carboxyl Butyl Chitosan (Chitolam NB/101—marketed by Pilot Chemical Company, developed by Lamberti); and Dicarboxyethyl Chitosan (N-[(3′-hydroxy-2′,3′-dicarboxy)ethyl]-beta-D-(1,4)-glucosamine) (available from Amerchol as, e.g., CHITOLAM NB/101).

Useful nonionic polymers include PVP or Polyvinylpyrrolidone (PVP K-15, K-30, K-60, K-90, K-120—supplier ISP) (Luviskol K series 12, 17, 30, 60, 80, & 90—supplier BASF); PVP/VA (PVP/VA series S-630; 735, 635, 535, 335, 235—supplier ISP)(Luviskol VA); PVP/DMAPA acrylates copolymer (Styleze CC-10—supplier ISP); PVP/VA/Vinyl Propionate copolymer (Luviskol VAP 343 E, VAP 343 I, VAP 343 PM—supplier BASF); Hydroxylethyl Cellulose (Cellosize HEC—supplier Amerchol); and Hydroxylpropyl Guar Gum (Jaguar HP series -8, -60, -105, -120—supplier Rhône-Poulenc).

A wide variety of natural, semi-natural, and synthetic styling polymers are useful herein, see suitable styling polymers in encyclopedia of polymers and thickeners, Cosmetic & Toiletries, Volume 117, No. 12, December 2002, pages 67-120.

9. Crosslinked Silicone Elastomers

The personal care compositions of the present invention may comprise crosslinked silicone elastomers. Crosslinked silicone elastomers are present in an amount of from about 0.01% to about 15%, preferably from about 0.1% to about 10%, even more preferably from about 1% to about 5% by weight of the composition. These benefit agents provide hair alignment and softness (emollient) benefits to hair. Preferred compositions are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), Grant Industries (Gransil™ line of materials), and lauryl dimethicone/vinyl dimethicone crosspolymers supplied by Shin Etsu (e.g., KSG-31, KSG-32, KSG-41, KSG-42, KSG-43, and KSG-44). Cross-linked organopolysiloxane elastomers useful in the present invention and processes for making them are further described in U.S. Pat. No. 4,970,252; U.S. Pat. No. 5,760,116; U.S. Pat. No. 5,654,362; and Japanese Patent Application JP 61-18708, assigned to Pola Kasei Kogyo KK. Silicone elastomers of the type described in U.S. Pat. Nos. 5,412,004; 5,837,793; and 5,811,487, are also useful herein. Preferably the elastomers of the present invention are cured under anhydrous conditions or in an anhydrous environment.

10. Peralkylene Hydrocarbons

The present invention may include peraklylene hydrocarbon materials. These materials are a branched alk(en)yl material, of which the side-groups are —H, C₁₋₄ alk(en)yl groups or (—H or C₁₋₄ alk(en)yl) substituted saturated or unsaturated cyclic hydrocarbons, and wherein at least 10% by number of the side-groups are other than —H, more preferably from 25% to 75%, most preferably from 40% to 60%. Preferred alkyl side-groups are methyl groups.

Preferably the weight average molecular weight of the per-alk(en)yl hydrocarbon material is less than about 4200, preferably from about 180 to about 2500. Such low molecular weight per-alk(en)yl hydrocarbon materials are available for example from BP under the trade name Indopol, from Soltex under the tradename Solanes and from Chevron under the tradename Oronite OLOA.

It is also advantageous to control the particle size of the per-alk(en)yl hydrocarbon materials in order to maintain suitable conditioning characteristic of the composition. The combination of per-alk(en)yl hydrocarbon materials having a particle size from about 0.01μ to about 40μ and cationic deposition polymers, especially celluloses, allow for the conditioning aspects of the formula to be controlled and targeted towards a given consumer group. Through the use of low molecular weight per-alk(en)yl hydrocarbon materials, the need for large levels of expensive conditioning oils to mitigate the trade-offs traditionally associated with styling shampoos is significantly reduced.

Preferred per-alk(en)yl hydrocarbon materials are polymers of butene, isoprene, terpene and styrene, and copolymers of any combination of these monomers, such as butyl rubber (poly isobutylene-co-isoprene), natural rubber (cis-1,4-polyisoprene) and hydrocarbon resins such as mentioned in the Encyclopedia of Chemical Technology by Kirk & Ohmer (3rd edition vol 8, pp 852-869), for example aliphatic and aromatic petroleum resins, terpene resins etc. Especially preferred is the use of polymers which are soluble in the low molecular weight per-alk(en)yl hydrocarbon material or other solvent or carrier, if used.

Especially preferred are per-alk(en)yl hydrocarbon materials of the formula: 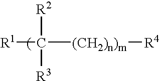
wherein:

n=0-3, preferably 1;

m=an integer such that the weight average molecular weight of the hydrocarbon is less than or equal to 4200.

R¹ is —H or a C₁₋₄ alkyl group; preferably methyl;

R² is a C₁₋₄ alkyl group; preferably methyl;

R³ is —H or a C₁₋₄ alkyl group; preferably —H or methyl 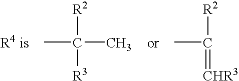
Especially preferred are polybutene materials of the formula: 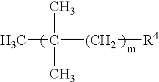
wherein R⁴ is

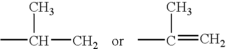

These materials are available from Presperse Inc. under the Permethyl trade name. The total level of per-alk(en)yl hydrocarbon material in the hair styling composition is preferably from about 0.01% to about 10%, more preferably from about 0.2% to about 5% even more preferably from about 0.2% to about 2% by weight of the composition.

11. Other Ingredients

The compositions herein can contain a variety of other optional components suitable for rendering such compositions more cosmetically or aesthetically acceptable or to provide them with additional usage benefits. Optional ingredients may be found in either the conditioning phase or the benefit phase. Such conventional optional ingredients are well-known to those skilled in the art.

A wide variety of additional ingredients can be formulated into the present composition. These include: other conditioning agents; viscosity modifiers such as alkanolamides and methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the conditioning compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Other optional hair and skin benefit ingredients include carboxylic acid which is hydroxylated in the a position (which compound is also referred to as an α-(alpha) hydroxyl acid) or a derivative thereof. Acid derivatives, as defined herein, are associated salts (salts with organic bases or alkali metal, for example) or lactides (obtained, for example, by autiesterification of α-hydroxy acid molecules). Examples of such compounds are, citric acid, lactic acid, methallactic acid, phenyllactic acid, malic acid, mandelic acid, glycolic acid, benzylic acid, and 2-hydroxycaprylic acid.

Additional hair and skin benefit agents include ceramides or glycoceramides. Ceramides are described in Arch. Dermatol, Vol 123, 1381-1384, 1987, or those described in French Patent FR-2,673,179; fatty acid polyesters such as, sucrose pentalaurate, sucrose tetraoleate, sucrose pentaerucate, sucrose tetraerucate, sucrose pentatallowate, sucrise triapeate, sucrose tetrapeate, sucrose pentarapeate, sucrose tristearate, and sucrose pentastearate, and mixtures thereof; polypeptides and amino acids consisting of basic amino acids, particularly arginine.

The compositions optionally comprise a colorant or pigment. Preferably, the colorant comprises metal ions. Preferably, the colorant is free of barium and aluminum ions which allows for improved lamellar phase stability. The colorant preferably maintains UV stability.

The colorants for use in the compositions are selected from the group consisting of organic pigments, inorganic pigments, interference pigments, lakes, natural colorants, pearlescent agents, dyes (including, for example, water-soluble, non-soluble, oil-soluble), carmines, and mixtures thereof. Non-limiting examples of colorants include: D&C Red 30 Talc Lake, D&C Red 7 Calcium Lake, D&C Red 34 Calcium Lake, Mica/Titanium Dioxide/Carmine Pigments (Clorisonne Red from Engelhard, Duocrome RB from Engelhard, Magenta from Rona, Dichrona RB from Rona), Red 30 Low Iron, D&C Red Lake Blend of Lake 27 & Lake 30, FD&C Yellow 5 Lake, Kowet Titanium Dioxide, Yellow Iron Oxide, D&C Red 30 Lake, D&C Red 28 Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake, Blue # 1, Orange # 4, Red # 4, Yellow # 5, and mixtures of these colorants.

### Density Matching

To further improve stability under stress conditions such as high temperature and vibration, it is preferable to adjust the densities of the separate phases such that they are substantially equal. This is known as density matching. To achieve density matching, low density microspheres may be added to the denser phase of the composition. The low density microspheres employed to reduce the overall density of the conditioning phase are particles having a density lower than about 0.7 g/cm³, preferably less than about 0.2 g/cm³, more preferably less than about 0.1 g/cm³, even more preferably less than about 0.05 g/cm³. The low density microspheres generally have a diameter less than about 200 μm, preferably less than about 100 μm, even more preferably less than about 40 μm. Preferably, the density difference between the conditioning phase and the benefit phase is less than about 0.30 g/cm³, preferably less than about 0.15 g/cm³, more preferably, the density difference is less than about 0.10 g/cm³, even more preferably, the density difference is less than about 0.05 g/cm³, and even more preferably, the density difference is less than about 0.01 g/cm³.

The microspheres are produced from any appropriate inorganic or organic material compatible with a use on the skin that is nonirritating and nontoxic.

Expanded microspheres made of thermoplastic material are known, and may be obtained, for example, according to the processes described in Patents and Patent Applications EP-56219, EP-348372, EP-486080, EP-320473, EP-112807 and U.S. Pat. No. 3,615,972.

The internal cavity of expanded hollow microspheres contains a gas, which can be a hydrocarbon such as isobutane or isopentane or alternatively air. Among hollow microspheres which can be used, special mention may be made of those marketed under the brand name EXPANCEL® (thermoplastic expandable microspheres) by the Akzo Nobel Company, especially those of DE (dry state) or WE (hydrated state) grade. Examples include: Expancel® 091 DE 40 d30; Expancel® 091 DE 80 d30; Expancel® 051 DE 40 d60; Expancel® 091 WE 40 d24; Expancel® 053 DE 40 d20.

Representative microspheres derived from an inorganic material, include, for instance, “Qcel® Hollow Microspheres” and “EXTENDOSPHERES™ Ceramic Hollow Spheres”, both available from the PQ Corporation. Examples are: Qcel® 300; Qcel® 6019; Qcel® 6042S.

Just as low density microspheres can be added to the denser phase of the present invention to improve vibrational stability, high density materials can be added to the less dense phase to increase its density having the same impact on stability.

The density of each phase is measured by a Pycnometer. Density is calculated in g/cc units. In matching densities, the densities of the two phases must not be substantially different and should preferably be within a range of +/−15%, more preferably within a range of +/−10%, even more preferably within a range of +/−5%.

### Method of Use

The personal care compositions of the present invention are used in conventional ways to provide conditioning and other benefits. Such method of use depends upon the type of composition employed but generally involves application of an effective amount of the product to the hair or skin, which may then be rinsed from the hair or skin (as in the case of hair rinses) or allowed to remain on the hair or skin (as in the case of gels, lotions, and creams). “Effective amount” means an amount sufficient enough to provide a dry combing benefit. In general, from about 1 g to about 50 g is applied to the hair on the scalp. The composition is distributed throughout the hair or skin, typically by rubbing or massaging the hair, scalp, or skin. Preferably, the composition is applied to wet or damp hair prior to drying of the hair. After such compositions are applied to the hair, the hair is dried and styled in accordance with the preference of the user. In the alternative, the composition is applied to dry hair, and the hair is then combed or styled in accordance with the preference of the user. The personal care compositions are useful in delivering conditioning benefits to hair or skin, and/or delivering hair styling benefits to hair or skin, and/or delivering hair coloring benefits to hair or skin by topically applying an effective amount of the composition onto hair or skin and rinsing said hair or skin with water. For some applications, the rinsing step can be optional.

### Method of Making

The personal care compositions of the present invention may be prepared by any known or otherwise effective technique, suitable for making and formulating the desired multi-phase product form. It is especially effective to combine toothpaste-tube filling technology with a spinning stage design. Specific non-limiting examples of such methods as they are applied to specific embodiments of the present invention are described in the following examples.

### Non Limiting Examples

The compositions illustrated in the following Examples exemplify specific embodiments of the compositions of the present invention, but are not intended to be limiting thereof. Other modifications can be undertaken by the skilled artisan without departing from the spirit and scope of this invention. These exemplified embodiments of the composition of the present invention provide enhanced deposition of the personal care composition due to enhanced coacervate formation.

The compositions illustrated in the following Examples are prepared by conventional formulation and mixing methods, an example of which is described above. All exemplified amounts are listed as weight percents and exclude minor materials such as diluents, preservatives, color solutions, imagery or conceptual ingredients, botanicals, and so forth, unless otherwise specified.

Examples 1-4

| | Example 1 | Example 2 | Example 3 | Example 4 |
|---|---|---|---|---|
| Conditioning Phase Composition | | | | |
| Stearamidopropyldimethylamine (1) | 2.0 | 2.0 | 3.0 | 2.0 |
| L-Glutamic acid (2) | 0.64 | 0.64 | 0.96 | 0.64 |
| Cetyl alcohol (3) | 2.5 | 3.75 | 3.75 | 2.5 |
| Stearyl alcohol (4) | 4.5 | 6.75 | 6.75 | 4.5 |
| Dimethicone blend (5) | — | — | 6.3 | — |
| Dimethicone/Cyclomethicone blend (6) | 4.2 | 4.2 | — | 4.2 |
| Benzyl alcohol (7) | 0.4 | 0.4 | 0.4 | 0.4 |
| EDTA (8) | 0.1 | 0.1 | 0.1 | 0.1 |
| Kathon CG (9) | 0.03 | 0.03 | 0.03 | 0.03 |
| Panthenyl Ethyl Ether (10) | 0.05 | 0.06 | 0.06 | 0.05 |
| Panthenol (11) | 0.09 | 0.09 | 0.05 | 0.09 |
| Perfume (12) | 0.25 | 0.20 | 0.25 | 0.25 |
| Deionized Water | qs | qs | qs | qs |
| Benefit Phase Composition | | | | |
| Versagel MD 1600 (13) | 97 | — | — | — |
| Versagel ME 750 (14) | — | 98 | — | — |
| Versagel ME-1600 (15) | — | — | 95 | — |
| Amino silicone (16) | 2.9 | — | — | — |
| Amino, polyol-silicone (17) | — | 1.9 | 4.9 | — |
| Dimethicone (41) | — | — | — | 100 |
| Pigment (from Rona) | 0.1 | 0.1 | 0.1 | — |
| Ratio Conditioning Phase/Benefit | 80/20 | 80/20 | 70/30 | 96/4 |
| Phase | | | | |

(1) supplied by Inolex under trade name Lexamine S-13
(2) supplied by Ajinomoto
(3) supplied by Procter & Gamble
(4) supplied by Procter & Gamble
(5) supplied by GE Silicones as a blend of dimethicone having a viscosity of 18,000,000 mPs · s and dimethicone having a viscosity of 200 mPa · s
(6) supplied by GE Silicone as a blend of dimethicone having a viscosity of 18,000,000 mPa.s and cyclopentasiloxane
(7) supplied by Haarman & Reimer
(8) supplied by BASF
(9) supplied by Rohm & Haas
(10) supplied by Roche
(11) supplied by Roche
(12) supplied by Firmenich
(13) supplied by Penreco
(14) supplied by Penreco
(15) supplied by Penreco
(16) supplied by Wacker as ADM1100
(17) supplied by Dow Corning Corporation as reference number 17828-137
(41) supplied Dow Corning (a high molecular weight dimethicone with a viscosity of about 300,000 cs.

In the conditioning phase compositions of examples 1 to 4, mix water, stearamidopropyldimethylamine, and L-glutamic acid at a temperature above 70° C. Then, add cetyl alcohol, stearyl alcohol, and benzyl alcohol with agitation. Cool down below 60° C., then add silicones, kathon, EDTA, panthenyl ethyl ether, panthenol and perfume with agitation. Then, cool down to about 30° C.

In the benefit phase compositions of examples 1 to 3, weigh the required quantity of an appropriate Versagel into a container and heat it to 40-50° C. while stirring. Then, add the other ingredients until a homogeneous mixture is obtained.

In the benefit phase composition of example 4, use dimethicone as received.

Density match the conditioning and benefit phases within 0.05 g/cm³. Combine these phases by first placing the separate phases in separate storage tanks having a pump and a hose attached. Then, pump the phases in predetermined amounts into a single combining section. Next, move the phases from the combining sections into blending sections and mix the phases in the blending section such that the single resulting product exhibits a distinct pattern of phases. Select the pattern from the group consisting of striped, marbled, geometric, and mixtures thereof. Next, pump the product that was mixed in the blending section via a hose into a single nozzle into a spinning container, and fill the container from the bottom to the top with the resulting product.

Examples 5-7

| Conditioner Phase Composition | Example 5 | Example 6 | Example 7 |
|---|---|---|---|
| Behetrimonium Chloride (18) | 3.38 | 2.25 | 4.05 |
| Cetyl alcohol | 2.32 | 1.86 | 2.5 |
| Stearyl alcohol | 4.18 | 4.64 | 4.5 |
| Polysorbate-60 (19) | 0.2 | 0.2 | 0.1 |
| Amino-silicone (16) | 2.0 | 0.5 | |
| Amino-, polyol-silicone (17) | — | — | 2.0 |
| Benzyl alcohol | 0.4 | 0.4 | 0.4 |
| EDTA | 0.13 | 0.13 | 0.13 |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | 0.014 | 0.014 |
| Isopropyl alcohol | 0.9 | — | 0.9 |
| Pigment (20) | 0.08 | 0.08 | 0.08 |
| Perfume | 0.5 | 0.5 | 0.5 |
| Deionized Water | qs | qs | Qs |
| Benefit Phase Composition | | | |
| Petrolatum (21) | 70 | | 80 |
| Dimethicone (6) | — | 70 | — |
| PEG-30 Dipolyhydroxystreate (22) | 1.0 | 1.5 | 2.0 |
| NaCl | 2.0 | 1.0 | 2.0 |
| Water | qs | qs | qs |
| Ratio Conditioning Phase/Benefit | 80/20 | 80/20 | 70/30 |
| Phase | | | |

(18) supplied by Clariant
(19) supplied by ICI as Tween 60
(20) supplied by Rona
(21) supplied by Witco
(22) supplied by Uniqima

The conditioning phase composition is made according to the methodology described for conditioning phase compositions of examples 1 to 4.

Prepare the benefit phase compositions by adding Petrolatum or Dimethicone into a vessel. Then, heat the vessel to 185° F. and then add Arlacel P135. Then, slowly add water containing appropriate salt or internal phase ingredients into the vessel with agitation until a homogeneous formulation is obtained.

If required, densities of two phases are matched and the product containing both phases as prepared according to the method described above for examples 1 to 4.

Examples 8-10

| Conditioning Phase Composition | Example 8 | Example 9 | Example 10 |
|---|---|---|---|
| Stearamidopropyldimethylamine | 2.0 | 2.0 | 3.0 |
| (1) | | | |
| L-Glutamic acid (2) | 0.64 | 0.64 | 0.96 |
| Cetyl alcohol (3) | 2.5 | 3.75 | 3.75 |
| Stearyl alcohol (4) | 4.5 | 6.75 | 6.75 |
| Dimethicone blend (5) | — | — | 6.3 |
| Dimethicone/Cyclomethicone | 4.2 | 4.2 | |
| blend (6) | | | |
| Benzyl alcohol (7) | 0.4 | 0.4 | 0.4 |
| EDTA (8) | 0.1 | 0.1 | 0.1 |
| Kathon CG (9) | 0.03 | 0.03 | 0.03 |
| Panthenyl Ethyl Ether (10) | 0.05 | 0.06 | 0.06 |
| Panthenol (11) | 0.09 | 0.09 | 0.05 |
| Perfume (12) | 0.25 | 0.20 | 0.25 |
| Deionized Water | qs | qs | qs |
| Benefit Phase Composition | | | |
| Dipropylene Glycol | 70 | — | — |
| Dibenzoate (24) | | | |
| Dimethicone (5) | — | 80 | — |
| 7-3105 Petrolatum HIP | — | — | 30 |
| Emulsion (25) | | | |
| Polyoxyethylene (20) | — | 1.0 | — |
| monolaurate (26) | | | |
| Plantaren 2000 (27) | 5.0 | — | — |
| Glycerin (28) | 5.0 | 0.75 | — |
| Water | qs | qs | — |
| Ratio Conditioning Phase/Benefit | 70/30 | 70/30 | 70/30 |
| Phase | | | |

(24) supplied by Finetex as Finsolv PG-22
(25) supplied by Dow Corning - used as a benefit phase
(26) supplied by Uniqema as Tween 20
(27) supplied by Cognis
(28) supplied by Procter&Gamble Chemicals

The conditioning phase composition is made according to the methodology described for conditioning phase compositions of examples 1 to 4.

Prepare the benefit phase compositions by mixing surfactant (Plantarn 2000, Tween 20) in the continuous phase (glycerin and/or water). Heat the batch to 50° C. and then slowly add oils (dipropylene glycol dibenzoate, dimethicone). Stir the mixture until homogeneous.

Examples 11-13

| | Example | Example | Example |
|---|---|---|---|
| Conditioner Phase Composition | 11 | 12 | 13 |
| Behetrimonium Chloride (18) | 3.38 | 2.25 | 4.05 |
| Cetyl alcohol | 2.32 | 1.86 | 2.5 |
| Stearyl alcohol | 4.18 | 4.64 | 4.5 |
| Polysorbate - 60 (19) | 0.2 | 0.2 | 0.1 |
| Amino-silicone (16) | 2.0 | 0.5 | |
| Amino-, polyol-silicone (17) | — | — | 2.0 |
| Benzyl alcohol | 0.4 | 0.4 | 0.4 |
| EDTA | 0.13 | 0.13 | 0.13 |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | 0.014 | 0.014 |
| Isopropyl alcohol | 0.9 | — | 0.9 |
| Pigment (20) | 0.08 | 0.08 | 0.08 |
| Perfume | 0.5 | 0.5 | 0.5 |
| Deionized Water | qs | qs | Qs |
| Benefit Phase Composition | | | |
| Silicone DC556 (29) | 27 | — | — |
| SEFA Cottonate (30) | — | 24 | — |
| KSG-310 Silicone (31) | — | — | 30 |
| Abil EM90 (32) | 3 | — | — |
| Poly Eth 617A (33) | — | 3 | — |
| PEG-30 Dipolyhydroxystreate (22) | — | 3 | — |
| NaCl | 1.0 | 1.0 | 1.0 |
| Water | qs | qs | qs |
| Ratio Conditioning Phase/Benefit | 70/30 | 80/20 | 80/20 |
| Phase | | | |

(29) supplied by Dow Corning
(30) supplied by P&G
(31) supplied by Shin Etsu
(32) supplied by Goldschmidt
(33) supplied by Honeywell

The conditioning phase composition is made according to the methodology described for conditioning phase compositions of examples 1 to 4.

Prepare the benefit phase compositions by dissolving salt in water. In a separate container, mix the oils with the appropriate surfactant (Abil EM90, Poly Eth 617A, PEG-30 Dipolyhydroxystreate). Slowly add water phase into oil phase and stir until a homogeneous mixture is obtained.

Examples 14

| Conditioning Phase Composition | Example 14 |
|---|---|
| Stearamidopropyldimethylamine | 1.0 |
| Cetyl alcohol | 0.96 |
| Stearyl alcohol | 0.64 |
| Dimethicone/Cyclomethicone blend | 4.2 |
| Benzyl alcohol | 0.4 |
| Quaternium-18 (34) | 0.75 |
| PEG-2M (Polyox WAR N-10) (35) | 0.5 |
| Emulsifying Wax (Polawax NF) (36) | 0.5 |
| Hydroxyethylcellulose (HEC) (37) | 0.25 |
| Glyceryl Monostearate (GMS) (38) | 0.25 |
| Oleyl Alcohol (39) | 0.25 |
| Citric Acid (40) | 0.13 |
| EDTA | 0.1 |
| Kathon CG | 0.03 |
| Panthenyl Ethyl Ether | 0.05 |
| Panthenol | 0.09 |
| Perfume | 0.25 |
| Deionized Water | qs |
| Benefit Phase Composition | |
| Versagel MD 1600 (13) | 97 |
| Amino silicone (16) | 2.9 |
| Pigment (from Rona) | 0.1 |
| Ratio Conditioning Phase/Benefit Phase | 40/60 |

(34) supplied by Degussa as Varisoft DHT
(35) supplied by Amerchol as Polyox WSR N-10
(36) supplied by Croda
(37) supplied by Aqualon as Natrosol 250 HHR
(38) supplied by Uniqema as Arlacel 129
(39) supplied by Croda as Novol
(40) supplied by Roche Vitamins

Prepare the conditioning phase of example 16 by adding PEG-2M and HEC to hot water with agitation. Add Stearamidopropyldimethylamine, Cetyl alcohol, Stearyl alcohol, Benzyl alcohol, Polawax, and Oleyl Alcohol at a temperature above 80° C. Cool down below about 60° C., then add silicones, kathon, EDTA, panthenyl ethyl ether, panthenol and perfume with agitation. Then, cool down to about 30° C.

In the benefit phase compositions of examples 14, weigh the required quantity of Versagel into a container and heat it to 40-50° C. while stirring. Then, add the other ingredients until a homogeneous mixture is obtained.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A multi-phase personal care composition comprising:
a. at least one conditioning phase, wherein said conditioning phase is a fatty compound-cationic surfactant; and
b. at least one benefit phase selected from the group consisting of high internal phase water-in-oil emulsions, hydrophobic gel networks, hydrophobic oil phases, and a silicone or silicone gel;
wherein said at least one conditioning phase and said at least one benefit are visually distinct phases that are packaged in physical contact with one another.

2. The multi-phase personal care composition of claim 1 wherein a visible pattern or arrangement of the visually distinct phases is substantially unchanged for a period of at least about 180 days, when said at least one conditioning phase and said at lease one benefit phase are sitting undisturbed in physical contact with one another at ambient conditions.

3. The multi-phase personal care composition of claim 1 wherein said at least one conditioning phase and said at least one benefit phase are visually distinct phases due to inclusion of a colorant in said at least one conditioning phase and/or in said at least one benefit phase.

4. The multi-phase personal care composition of claim 1 wherein said at least one conditioning phase or said at least one benefit phase or both said at least one conditioning phase and said at least one benefit phase further comprise a density modifier.

5. The multi-phase personal care composition of claim 1 wherein said at least one conditioning phase or said at least one benefit phase or both said at least one conditioning phase and said at least one benefit phase further comprise an aqueous carrier.

6. The multi-phase personal care composition of claim 1 wherein said at least one conditioning phase or said at least one benefit phase or both said at least one conditioning phase and said at least one benefit phase further comprise at least one additional component selected from the group consisting of humectants; solutes; water soluble nonionic polymers; hair coloring agents/dyes; cationic polymer conditioning agents; silicone conditioning agents; anti-dandruff agents; particles; styling polymers; crosslinked silicone elastomers; and peralkylene hydrocarbons.

7. The multi-phase personal care composition of claim 1 wherein said at least one conditioning phase is present in an amount of from about 1% to about 99% by weight of the composition.

8. The multi-phase personal care composition of claim 1 wherein said at least one conditioning phase is present in an amount of from about 3% to about 97% by weight of the composition.

9. The multi-phase personal care composition of claim 1 wherein said at least one benefit phase is present in an amount of from about 1% to about 95% by weight of the composition.

10. The multi-phase personal care composition of claim 1 wherein said at least one benefit phase is present in an amount of from about 5% to about 90% by weight of the composition.

11. The multi-phase personal care composition of claim 1 wherein said at least one benefit phase is present in an amount of from about 10% to about 80% by weight of the composition.

12. The multi-phase personal care composition of claim 1 wherein said at least one benefit phase, said at least one conditioning phase, or both said at least one benefit phase and said at least one conditioning phase are visibly clear.

13. The multi-phase personal composition of claim 1 wherein said visually distinct phases form a pattern selected from group consisting of striped, geometric, marbled, and combinations thereof.

14. The multi-phase personal care composition of claim 1 wherein the density difference between said at least one conditioning phase and said at least one benefit phase is less than about 0.30 g/cm³.

15. The multi-phase personal care composition of claim 1 wherein the density difference between said at least one conditioning phase and said at least one benefit phase is less than about 0.05 g/cm³.

16. The multi-phase personal care composition of claim 1 wherein the viscosity of said at least one conditioning phase and said at least one benefit phase are in the range of from about 10,000 to about 200,000,000 centipoise at stress measurements of from about 1 to about 20 pascals at about 25° C.

17. The multi-phase personal care composition of claim 1 wherein the viscosity of said at least one conditioning phase and said at least one benefit phase are in the range of from about 100,000 to about 100,000,000 centipoise at stress measurements of from about 1 to about 20 pascals at about 25° C.

18. The multi-phase personal care composition of claim 1 wherein said at least one benefit phase is a water-in-oil emulsion, and wherein the water component of the emulsion comprises a water-soluble dye.

19. The multi-phase personal care composition of claim 1 wherein said at least one benefit phase is a hydrophobic oil phase that comprises an oil-soluble dye.

20. A method of delivering conditioning benefits to hair or skin, said method comprising the steps of:
a) topically applying an effective amount of a composition according to claim 1 onto said hair or skin; and
b) optionally rinsing said hair or skin with water.

21. A method of delivering hair styling benefits to hair or skin, said method comprising the steps of:
a) topically applying an effective amount of a composition according to claim 6 onto said hair or skin; and
b) optionally rinsing said hair or skin with water.

22. A method of delivering hair coloring benefits to hair or skin, said method comprising the steps of:
a) topically applying an effective amount of a composition according to claim 6 onto said hair or skin; and
b) rinsing said hair or skin with water.

23. A method of delivering anti-dandruff benefits, said method comprising the steps of:
a) topically applying an effective amount of a composition according to claim 6 onto hair or skin; and
b) rinsing said hair or skin with water.

24. A method of delivering volumizing benefits to hair, said method comprising the steps of:
a) topically applying an effective amount of a composition according to claim 6 onto said hair; and
b) optionally rinsing said hair with water.
